(19) United States
(12) Patent Application Publication
(10) Pub. No.: US 2025/0302553 A1
(43) Pub. Date: Oct. 2, 2025

(54) NAVIGATION UPDATES FOR MEDICAL SYSTEMS

(71) Applicant: AURIS HEALTH, INC., Santa Clara, CA (US)

(72) Inventors: Mali Shen, Sunnyvale, CA (US); Gang Dong, Redwood City, CA (US); Namita Anil Kumar, Redwood City, CA (US); Wennan Zhao, Brookfield, WI (US); Hedyeh Rafii-Tari, Mountain View, CA (US); Jeffrey Adam Kasten, San Mateo, CA (US)

(73) Assignee: AURIS HEALTH, INC., Santa Clara, CA (US)

(21) Appl. No.: 19/034,431

(22) Filed: Jan. 22, 2025

Related U.S. Application Data
(60) Provisional application No. 63/571,909, filed on Mar. 29, 2024.

(51) Int. Cl. A61B 34/00; A61B 34/10; A61B 34/20; A61B 90/00

(52) U.S. Cl. CPC A61B 34/25; A61B 2034/105; A61B 2034/107; A61B 2034/2065; A61B 2034/2072; A61B 2090/3764

(57) ABSTRACT

This disclosure provides methods, devices, and systems for planning and performing medical procedures. The present implementations more specifically relate to techniques for navigating an instrument to a target within an anatomy. In some aspects, a controller for a medical system may generate a graphical interface depicting a spatial relationship between the instrument and the target and update the graphical interface to depict an updated spatial relationship between the instrument and the target based on sensor data received via a sensor disposed on the instrument and image data captured by an imaging system external to the anatomy while the instrument is disposed within the anatomy. More specifically, the controller may determine a mapping between a sensor space and an image space based on the sensor data and the image data and may determine the updated spatial relationship based on the mapping between the sensor space and the image space.

### CROSS-REFERENCE TO RELATED APPLICATIONS

This application claims priority and benefit under 35 U.S.C. § 119(e) to U.S. Provisional Patent Application No. 63/571,909, filed Mar. 29, 2024, which is incorporated herein by reference in its entirety.

### TECHNICAL FIELD

This disclosure relates generally to medical systems, and specifically to intraoperative navigation updates for medical systems.

### DESCRIPTION OF RELATED ART

Many medical procedures include steps that can be performed pre-operation (also referred to as a “preoperative phase”), intra-operation (also referred to as an “intraoperative phase”), or post-operation (also referred to as a “postoperative phase”). For example, during a preoperative phase, an imaging system may be used to scan or otherwise capture images or video of a patient's anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. The images may be used, during an intraoperative phase, to help guide or navigate a medical instrument to a target (also referred to as a “treatment site”) within the patient's anatomy. However, images acquired during a preoperative phase may not accurately reflect a spatial relationship between the medical instrument and the target during an intraoperative phase. For example, among various other factors, preoperative images are often acquired several days (or even weeks) before the intraoperative phase, such that changes in the patient's anatomy may cause deviations in the spatial positioning of the target. Thus, there is a need to provide more accurate information about the spatial relationship between the medical instrument and the target during the intraoperative phase.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for navigating an instrument within an anatomy of a patient. The method includes steps of generating a graphical interface depicting a spatial relationship between the instrument and a target within the anatomy; receiving first image data captured by a first imaging system external to the anatomy while the instrument is disposed within the anatomy; receiving first sensor data via one or more sensors associated with a sensor system, where the one or more sensors include at least a first sensor disposed on the instrument; determining a mapping between a first coordinate space associated with the first imaging system and a second coordinate space associated with the sensor system based at least in part on the first image data and the first sensor data; and updating the graphical interface to depict an updated spatial relationship between the instrument and the target based at least in part on the mapping between the first coordinate space and the second coordinate space.

Another innovative aspect of the subject matter of this disclosure can be implemented in a controller for a medical system, including a processing system and a memory. The memory stores instructions that, when executed by the processing system, cause the controller to generate a graphical interface depicting a spatial relationship between the instrument and a target within the anatomy; receive first image data captured by a first imaging system external to the anatomy while the instrument is disposed within the anatomy; receive first sensor data via one or more sensors associated with a sensor system, where the one or more sensors include at least a first sensor disposed on the instrument; determine a mapping between a first coordinate space associated with the first imaging system and a second coordinate space associated with the sensor system based at least in part on the first image data and the first sensor data; and update the graphical interface to depict an updated spatial relationship between the instrument and the target based at least in part on the mapping between the first coordinate space and the second coordinate space.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows example components of the control system and the robotic system of FIG. 1, according to some implementations.

FIG. 3 shows a block diagram of an example localization system, according to some implementations.

FIG. 4 shows a block diagram of an example navigation system, according to some implementations.

FIG. 5 shows an example spatial relationship between a skeleton image space, a sensor space, and an intraoperative image space.

FIG. 6 shows an example update to target position relative to instrument pose.

FIG. 7 shows an example update to instrument pose relative to target position.

FIG. 8 shows example corrections to instrument pose and target position in their respective coordinate spaces and updates to a spatial relationship between the instrument and the target in a common coordinate space.

FIG. 9 shows a block diagram of an example controller for a medical system, according to some implementations.

FIG. 10 shows an illustrative flowchart depicting an example operation for navigating an instrument within an anatomy, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for case of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures include a preoperative phase that precedes an intraoperative phase. During the preoperative phase, for some medical procedures, an imaging system may be used to scan or otherwise capture images or video of at least a portion of a patient's anatomy. For example, a computed tomography (CT) scanner may be used to acquire tomographic images (also referred to as “tomograms” or “CT scans”) of a patient's lungs during the preoperative phase for a bronchoscopy. A tomogram is a cross-section or slice of a three-dimensional (3D) volume. For example, multiple tomograms can be stacked or combined to recreate the 3D volume (such as a 3D model of the patient's lungs). Thus, tomograms can be used to detect a precise location or position (in 3D space) of a nodule or target in the patient's lungs. During the intraoperative phase, for some medical procedures, a medical system may use the preoperative images to generate a graphical interface for navigating a medical instrument within the patient's anatomy. For example, during a bronchoscopy, the medical system may detect a pose of an endoscope (such as a position and orientation of the scope in 3D space) based on sensor data received via an electromagnetic (EM) sensor disposed on the tip of the scope and map the pose of the endoscope to a 3D model of the patient's lungs depicted by the tomograms.

Accordingly, the graphical interface may depict a spatial relationship between the medical instrument and the target within the anatomy based on the sensor data and the image data. However, images acquired during a preoperative phase may not accurately reflect the spatial relationship between the medical instrument and the target during an intraoperative phase. For example, changes in the patient's anatomy or the medical environment can cause the spatial relationship between the endoscope and target to deviate from what is depicted by the graphical interface at any given time, which can lead to inaccurate navigation. Example factors include EM distortion, poor registration (or mapping) between the sensor space and the image space associated with the preoperative scans (also referred to as the “preoperative image space”), outdated preoperative scans, and anatomical deformations, among other examples. Aspects of the present disclosure recognize that some modern imaging technologies (such as cone beam CT) can be used to scan a patient's anatomy during an intraoperative phase. In some aspects, a medical system may capture updated images of the patient's anatomy during the intraoperative phase and use the updated image data to improve the representation of the spatial relationship between the medical instrument and the target.

The updated image data and sensor data are often associated with different coordinate spaces. Thus, in some implementations, the medical system may “register” the updated image space with the sensor space to facilitate real-time navigation. As used herein, the term “registration” refers to a mapping or transformation between different coordinate spaces. For example, a medical system may register an imaging system used for capturing images of a patient's anatomy (such as a cone beam CT scanner) with a sensor system used for tracking a pose of a medical instrument within the anatomy (such as an EM field generator) by determining a mapping or spatial transformation that maps any point or vector in the image space to a respective point or vector in the sensor space (such as a transformation matrix). The terms “mapping,” “transformation,” “spatial transformation,” and “registration matrix,” may be used interchangeably herein. The terms “respective” and “corresponding” also may be used interchangeably herein.

Although certain aspects of the present disclosure are described in detail herein in the context of bronchoscopy, it should be understood that the systems and techniques of the present disclosure may be applicable to any medical procedure. Example medical procedures may include minimally invasive procedures (such as laparoscopy), non-invasive procedures (such as endoscopy), therapeutic procedures, diagnostic procedures, percutaneous procedures, and non-percutaneous procedures, among other examples. Example endoscopic procedures include bronchoscopy, ureteroscopy, gastroscopy, nephroscopy, and nephrolithotomy, among other examples. The terms “scope,” “endoscope,” “catheter,” and “instrument” may be used interchangeably herein.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples)

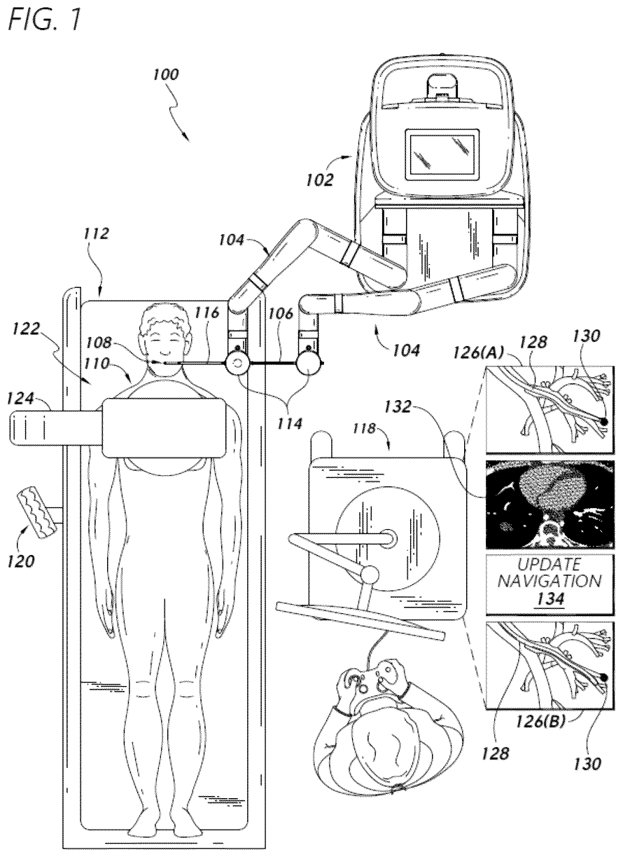

FIG. 1 shows an example medical system 100 (also referred to as a “surgical medical system” or a “robotic medical system”), according to some implementations. As shown in FIG. 1, the medical system 100 may be arranged for diagnostic or therapeutic bronchoscopy. The medical system 100 can include and utilize a robotic system 102 which can be implemented, for example, as a robotic cart. Although the medical system 100 is shown as including various cart-based systems or devices, the concepts disclosed herein can be implemented in any type of robotic system or arrangement, such as robotic systems employing rail-based components, table-based robotic end-effectors, or manipulators, among other examples. The robotic system 102 may include one or more robotic arms 104 (also referred to as “robotic positioners”) configured to position or otherwise manipulate a medical instrument 106 (such as a steerable endoscope or another elongate instrument). For example, the medical instrument 106 can be advanced through a natural orifice access point (such as the mouth 108 of a patient 110 positioned on a table 112) to deliver diagnostic or therapeutic treatment. Although described in the context of a bronchoscopy procedure, the medical system 100 also may be used to perform other types of medical procedures. Example suitable procedures include gastro-intestinal (GI) procedures, renal procedures, urological procedures, and nephrological procedures, among other examples.

With the robotic system 102 properly positioned, the medical instrument 106 can be inserted into the patient 110 robotically, manually, or a combination thereof. For example, the one or more robotic arms 104, or instrument drivers 114 coupled thereto, can control the medical instrument 106. In some implementations, the medical instrument 106 may be advanced within a sheath 116. For example, the sheath 116 may be coupled to, or controlled by, a robotic arm 104. In some implementations, the medical instrument 106 and the sheath 116 may each be coupled to a respective instrument driver from a set of instrument drivers 114. The instrument drivers 114 can be repositionable in space by manipulating the one or more robotic arms 104 into different angles or positions.

In the example of FIG. 1, the medical instrument 106 can be directed down the patient's trachea and lungs after insertion or advanced to a target destination or operative site. In some implementations, to enhance navigation through the patient's lung network or reach the desired target, the medical instrument 106 may be manipulated to telescopically extend from the outer sheath 116 to obtain enhanced articulation or greater bend radius. The use of separate instrument drivers 114 can allow the medical instrument 106 and sheath 116 to be driven independently of each other.

In some implementations, the medical instrument 106 may include an elongate member or shaft configured to be inserted or retracted, articulated, or otherwise moved within the anatomy. Further, in some implementations, the medical instrument 106 may include one or more imaging devices (such as cameras) positioned on a distal end of the elongate shaft or deployed through a working channel of the elongate shaft. The imaging devices can be configured to generate or capture image (or video) data or send the image data to another device or component. In some implementations, the medical instrument 106 may include an instrument base or one or more handles positioned at a proximal end of the medical instrument 106. The instrument base can be coupled to a manipulator (such as an end of a robotic arm 104). The instrument base can include one or more drive inputs coupled to one or more drive outputs of the manipulator, wherein the drive inputs or drive outputs act as an interface.

In some implementations, the medical instrument 106 may include a working channel configured to receive one or more other instruments or elements therein or provide other functionality. The working channel can extend axially, such as along the length of the medical instrument 106. Furthermore, the medical instrument 106 can include or be associated with one or more elongate movement members (such as pulls wires) that can extend from a proximal end through the elongate shaft to the distal end of the elongate shaft. The elongate movement members can be manipulated, such as by manipulators on the one or more robotic arms 104, to control actuation of the elongate movement members.

In some implementations, the medical instrument 106 may include one or more sensors, such as electromagnetic (EM) sensors, shape sensors (such as shape sensing fiber), accelerometers, gyroscopes, satellite-based positioning sensors (such as global positioning system (GPS) sensors), or radio-frequency (RF) transceivers, among other examples. The sensors can be configured to generate or produce sensor data or provide the sensor data to another device or component. The sensors can be disposed at a distal end of the elongate shaft or along a length of the elongate shaft. In some implementations, the medical instrument 106 may be configured to receive an elongate member or device through a working channel, wherein the elongate member includes one or more sensors along a length of the elongate member. One or more sensors on the medical instrument 106 may provide sensor data to control circuitry of the medical system 100, which is then used to determine a position, orientation, or shape of the medical instrument 106.

The medical system 100 can also include a control system 118 (also referred to as a “control tower” or “mobile tower”). The control system 118 can be communicatively coupled (such as via wired or wireless connections) to the robotic system 102 to control various aspects of the robotic system 102 (such as electronics, optics, sensors, or power) or one or more subsystems associated with the robotic system 102, such as a fluid management system (not shown). Placing such functionality in the control system 118 can allow for a smaller form factor of the robotic system 102 that may be more easily adjusted or re-positioned by an operator or user. Additionally, the division of functionality between the robotic system 102 and the control system 118 can reduce operating room clutter and facilitate efficient clinical workflow.

The medical system 100 can include an electromagnetic (EM) field generator 120, which is configured to broadcast or emit an EM field that can be detected by various EM sensors, such as a sensor disposed on the medical instrument 106. The EM field can induce small electric currents in coils of the EM sensors, which can be analyzed to determine a position, angle, or orientation of the EM sensors relative to the EM field generator 120. Although EM fields and EM sensors are described in many examples herein, position sensing systems or sensors can include various other types of position sensing systems or sensors, such as optical position sensing systems or sensors, image-based position sensing systems or sensors, among other examples.

The medical system 100 can further include an imaging system 122 (also referred to as an “imaging device”) configured to generate, provide, or send image data (also referred to as “images”) to another device or system. For example, the imaging system 122 can generate image data depicting an anatomy of the patient 110 and provide the image data to the control system 118, the robotic system 102, or another device. The imaging system 122 may include an emitter or energy source (such as an X-ray source) or a detector (such as an X-ray detector) mounted on a C-shaped arm support 124, which allows for flexibility in positioning around the patient 110 to capture images from various angles without moving the patient 110. Use of the imaging system 122 can provide visualization of internal structures or anatomy, which can be used for a variety of purposes, including navigation of the medical instrument 106 (such as by providing images of internal anatomy to a user) and localization of the medical instrument 106 (based on an analysis of image data), among other examples. In some aspects, the imaging system 122 may enhance the efficacy or safety of a medical procedure, such as a bronchoscopy, by providing clear, continuous visual feedback to the operating surgeon or team.

In some implementations, the imaging system 122 may be a mobile device configured to move around an environment. For example, the imaging system 122 can be positioned next to the patient 110 (as shown in FIG. 1) during a particular phase of a procedure and removed when the imaging system 122 is no longer needed. In some other implementations, the imaging system 122 may be part of the table 112 or other equipment in an operating environment. The imaging system 122 can be implemented as a Computed Tomography (CT) machine or system, X-ray machine or system, fluoroscopy machine or system, Positron Emission Tomography (PET) machine or system, PET-CT machine or system, CT angiography machine or system, Cone-Beam CT (CBCT) machine or system, three-dimensional rotational angiography (3DRA) machine or system, single-photon emission computed tomography (SPECT) machine or system, Magnetic Resonance Imaging (MRI) machine or system, Optical Coherence Tomography (OCT) machine or system, or ultrasound machine or system, among other examples. In some implementations, the medical system 100 may include different types of imaging systems that can be used or positioned over the patient 110 during different phases or portions of a procedure depending on the needs at that time.

In some implementations, the imaging system 122 may be configured to process multiple images (also referred to as “image data”) to generate a three-dimensional (3D) view or model. For example, the imaging device 122 can be implemented as a CT machine configured to capture or generate a series of images (also referred to as “tomograms) or image data representing two-dimensional (2D) cross-sections or slices of a 3D volume from different angles around the patient 110, and then use one or more algorithms to reconstruct these images or image data into a 3D model. The 3D model can be provided to the control system 118, robotic system 102, or another device, such as for processing or display.

In some implementations, image data from the imaging system 122 may be used to localize various elements, such as the medical instrument 106, a target within the anatomy, or specific anatomical features, among other examples. As used herein, the terms “localize,” “localization,” or “localizing” refer to any processes for determining or estimating a position (or location) and/or orientation (or heading), collectively referred to as the “pose,” of the instrument or the target (or any other element) within a given space or environment. For example, the control system 118 can be configured to provide navigation information during a procedure to assist a user navigating the medical instrument 106 within the anatomy to reach a target (such as a desired treatment site or location). In some implementations, a target can include a nodule, such as in the context of certain bronchoscopy procedures. To illustrate, the control system 118 can display a navigation view or graphical data 126 that includes an instrument indicator 128 representing the medical instrument 106, a target indicator 130 representing the target, and an anatomical map. The navigation data 126 (A) (such as initial navigation data) can be determined based on sensor data from a sensor of the medical instrument 106 (such as EM sensor data associated with the EM field generator 120), a map of the anatomy, or a location of the target. In some implementations, the map or location of the target may be determined based on preoperative data, such as data obtained during a preoperative procedure to find a target location or map the anatomy.

In some implementations, the navigation data 126 (A) may be dynamically updated based on image data 132 from the imaging system 122. For example, the control system 118 can receive the image data 132 and analyze the image data 132 to determine a current or actual spatial relationship between the medical instrument 106 and the target. In some implementations, the control system 118 may display the image data 132 to a user, receive user input indicating a position of the medical instrument 106 or a position of the target in the image data 132, and analyze the image data 132 based on the user input to determine the current spatial relationship. If the control system 118 determines that the navigation data 126 (A) incorrectly depicts the location of the medical instrument 106 (such as where the spatial relationship associated with the image data 132 is different than the spatial relationship associated with the navigation data 126 (A)), the control system 118 may update the navigation data 126 (A) at 134 and provide updated navigation data 126 (B) that reflects the current or near real-time position of the medical instrument 106 relative to the target or the map.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, the Internet, personal area networks (PANs), body area network (BANs), etc. In some examples, various communication interfaces can include wireless technology, such as Bluetooth, Wi-Fi, near-field communication (NFC), or the like. Furthermore, in some examples, the various components of the medical system 100 can be connected for data communication, fluid exchange, power exchange, and so on, via one or more support cables, tubes, connections, or the like.

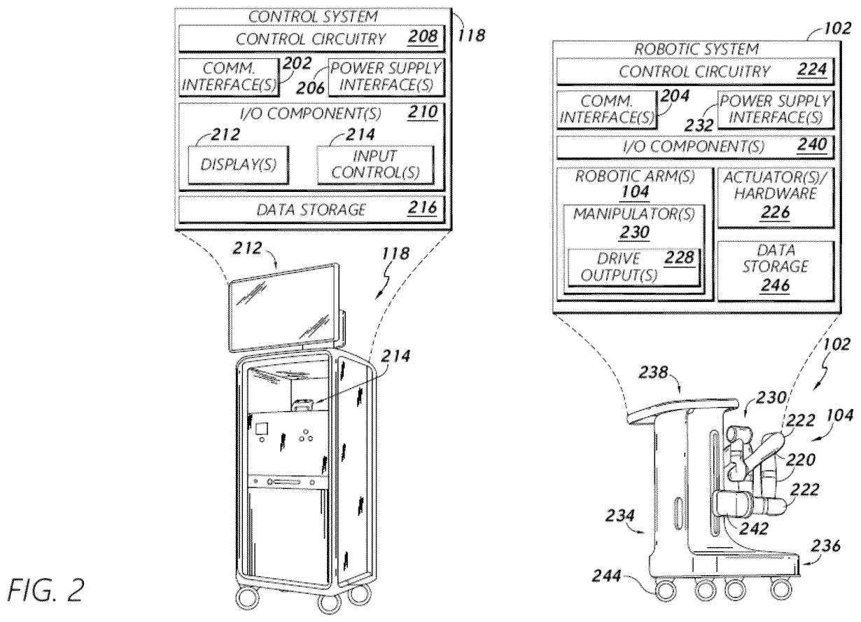

FIG. 2 shows example components of the control system 118 and the robotic system 102 of FIG. 1, according to some implementations. In the examples of FIG. 2, the control system 118 and the robotic system 102 are implemented as a tower and a robotic cart, respectively. However, the control system 118 and robotic system 102 can be implemented in other manners. The control system 118 can be coupled to the robotic system 102 and operate in cooperation therewith to perform a medical procedure. For example, the control system 118 can include communication interface(s) 202 for communicating with communication interface(s) 204 of the robotic system 102 via a wireless or wired connection (such as to control the robotic system 102). In some implementations, the control system 118 may communicate with the robotic system 102 to receive position or sensor data therefrom relating to the position of sensors associated with an instrument or member controlled by the robotic system 102. For example, the control system 118 may communicate with the EM field generator 120 to control generation of an EM field in an area around a patient. The control system 118 can further include one or more power supply interface(s) 206.

The control system 118 can include control circuitry 208 configured to cause one or more components of the medical system 100 to actuate or otherwise control any of the various system components, such as carriages, mounts, arms or positioners, medical instruments, imaging devices, position sensing devices, or sensors, among other examples. Further, the control circuitry 208 can be configured to perform other functions, such as cause display of information, process data, receive input, communicate with other components or devices, or any other function or operation described herein.

The control system 118 can further include one or more input or out (I/O) components 210 configured to assist a physician or others in performing a medical procedure. For example, the one or more I/O components 210 can be configured to receive input or provide output to enable a user to control or navigate the medical instrument 106, the robotic system 102, or other instruments or devices associated with the medical system 100. The control system 118 can include one or more displays 212 to provide, display or otherwise present various information regarding a procedure. For example, the one or more displays 212 can be used to present navigation information including a virtual anatomical model of anatomy with a virtual representation of a medical instrument, image data, or other information. The one or more I/O components 210 can include one or more user input control(s) 214, which can include any type of user input (or output) devices or device interfaces, such as one or more buttons, keys, joysticks, handheld controllers (such as video-game-type controllers), computer mice, trackpads, trackballs, control pads, sensors (such as motion sensors or cameras) that capture hand gestures and finger gestures, touchscreens, toggle (such as button) inputs, or interfaces or connectors therefore. In some implementations, such inputs can be used to generate commands for controlling one or more medical instruments, robotic arms, or other components.

The control system 118 can also include data storage 216 configured to store executable instruments (such as computer-readable instructions) that can be executed by the control circuitry 208 to cause the control circuitry 208 to perform various operations or functionality described herein. In some implementations, the data storage 216 also may store telemetry or runtime data (such as sensor data or image data) generated by the medical system 100 or otherwise captured or acquired during a medical procedure. In some implementations, two or more components of the control system 118 can be electrically or communicatively coupled to each other.

The robotic system 102 can include the one or more robotic arms 104 configured to engage with or control, for example, the medical instrument 106 or other elements or components to perform one or more aspects of a procedure. As shown in FIG. 2, each robotic arm 104 can include multiple segments 220 coupled to joints 222, which can provide multiple degrees of movement or freedom. The robotic system 102 can be configured to receive control signals from the control system 118 to perform certain operations, such as to position one or more of the robotic arms 104 in a particular manner or manipulate an instrument, among other examples. In response, the robotic system 102 can control, using control circuitry 224 thereof, actuators 226 or other components of the robotic system 102 to perform the operations. For example, the control circuitry 224 can control insertion or retraction, articulation, or roll of a shaft of the medical instrument 106 or other instrument by actuating one or more drive outputs 228 of a manipulator 230 (or end-effector) coupled to a base of a robotically-controllable instrument. The drive outputs 228 can be coupled to a drive input on an associated instrument, such as an instrument base of an instrument that is coupled to the associated robotic arm 104. The robotic system 102 also may include one or more power supply interfaces 232.

The robotic system 102 can include a support column 234, a base 236, or a console 238. The console 238 can provide one or more I/O components 240, such as a user interface for receiving user input or a display screen (or a dual-purpose device, such as a touchscreen) to provide the physician or user with preoperative or intraoperative data. The support column 234 can include an arm support 242 (also referred to as a “carriage”) for supporting the deployment of the one or more robotic arms 104. The arm support 242 can be configured to vertically translate along the support column 234. Vertical translation of the arm support 242 allows the robotic system 102 to adjust the reach of the robotic arms 104 to meet a variety of table heights, patient sizes, or physician preferences. The base 236 can include wheel-shaped casters 244 (also referred to as “wheels”) that allow the robotic system 102 to move around the operating room. After reaching the appropriate position, the casters 244 can be immobilized using wheel locks to hold the robotic system 102 in place during the procedure.

The joints 222 of each robotic arm 104 can each be independently-controllable or provide an independent degree of freedom available for instrument navigation. In some implementations, each robotic arm 104 may include seven joints that provide seven degrees of freedom, including “redundant” degrees of freedom. Redundant degrees of freedom can allow robotic arms 104 to be controlled to position their respective manipulators 230 at a specific position, orientation, or trajectory in space using different linkage positions and joint angles. This allows for the robotic system 102 to position or direct a medical instrument from a desired point in space while allowing the physician to move the joints 222 into a clinically advantageous position away from the patient to create greater access, while avoiding collisions.

The one or more manipulators 230 (or end-effectors) can be coupled to an instrument base or handle, which can be attached using a sterile adapter component. The combination of the manipulator 230 and instrument base, as well as any intervening mechanics or couplings (such as the sterile adapter), can be collectively referred to as the manipulator or a manipulator assembly. Manipulators or manipulator assemblies can provide power or control interfaces. Example interfaces may include connectors to transfer pneumatic pressure, electrical power, electrical signals, or optical signals from the robotic arm 104 to an instrument base. Manipulators or manipulator assemblies can be configured to manipulate medical instruments (such as surgical tools) using techniques including, for example, direct drives, harmonic drives, geared drives, belts or pulleys, or magnetic drives, among other examples.

The robotic system 102 can also include data storage 246 configured to store executable instruments (such as computer-readable instructions) that can be executed by the control circuitry 224 to cause the control circuitry 224 to perform various operations or functionality described herein. In some implementations, the data storage 216 also may store telemetry or runtime data (such as sensor data or image data) generated by the medical system 100 or otherwise captured or acquired during a medical procedure. In some implementations, two or more of the components of the robotic system 102 can be electrically or communicatively coupled to each other.

Data storage (including the data storage 216, data storage 246, or other data storage or memory) can include any suitable or desirable type of computer-readable media. For example, computer-readable media can include one or more volatile data storage devices, non-volatile data storage devices, removable data storage devices, or nonremovable data storage devices implemented using any technology, layout, or data structure(s) or protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Functionality described herein can be implemented by the control circuitry 208 of the control system 118 or the control circuitry 224 of the robotic system 102, such as by the control circuitry 208 or 224 executing instructions to cause the control circuitry 208 or 224 to perform the functionality. Control circuitry (including the control circuitry 208, control circuitry 224, or other control circuitry) can include circuitry embodied in a robotic system, control system or tower, instrument, or any other component or device. Control circuitry can include any collection of processors, processing circuitry, processing modules or units, chips, dies (such as semiconductor dies including one or more active or passive devices or connectivity circuitry), microprocessors, micro-controllers, digital signal processors, microcomputers, central processing units, field-programmable gate arrays, programmable logic devices, state machines (such as hardware state machines), logic circuitry, analog circuitry, digital circuitry, or any device that manipulates signals (analog or digital) based on hard coding of the circuitry or operational instructions.

Control circuitry referenced herein can further include one or more circuit substrates (such as printed circuit boards), conductive traces and vias, or mounting pads, connectors, or components. Control circuitry can further include one or more storage devices, which may be embodied in a single device, a plurality of devices, or embedded circuitry of a device. Such data storage can comprise read-only memory, random access memory, volatile memory, non-volatile memory, static memory, dynamic memory, flash memory, cache memory, data storage registers, or any device that stores digital information. In examples in which control circuitry includes a hardware or software state machine, analog circuitry, digital circuitry, or logic circuitry, data storage device(s) or register(s) storing any associated operational instructions can be embedded within, or external to, the circuitry comprising the state machine, analog circuitry, digital circuitry, or logic circuitry.

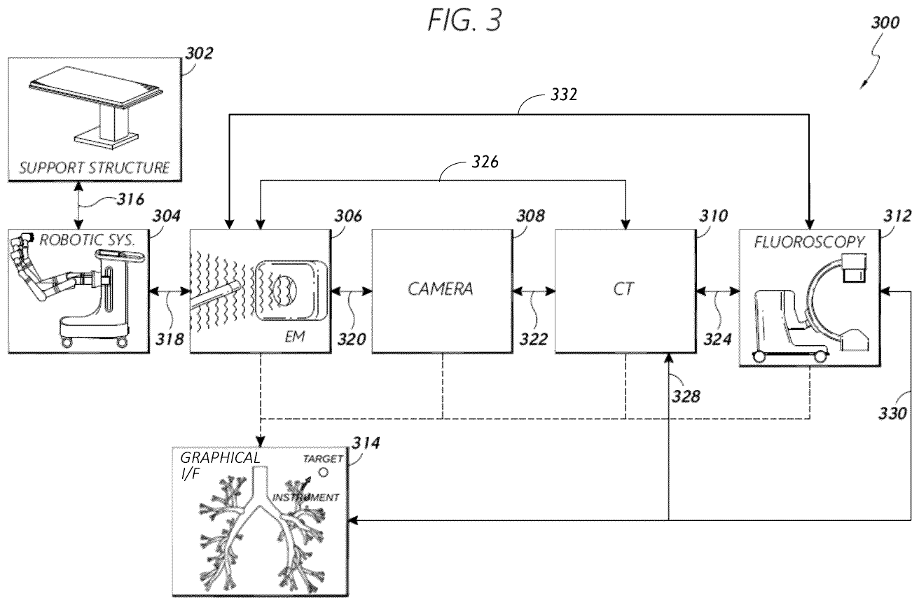

FIG. 3 shows a block diagram of an example localization system 300, according to some implementations. The localization system 300 includes various positioning or imaging systems or modalities 302-312 (also referred to as “subsystems”), which can be implemented to facilitate anatomical mapping, navigation, positioning, or visualization for procedures in accordance with one or more examples. For example, the various systems 302-312 can be configured to provide data for generating an anatomical map, determining a location of an instrument, determining a location of a target, or performing other techniques.

Each of the systems 302-312 can be associated with a respective coordinate space (also referred to as a “position coordinate frame”) or can provide data or information relating to instrument or anatomy locations, wherein registering the various coordinate spaces to one another can allow for integration of the various systems to provide mapping, navigation, or instrument visualization. For example, registering a first modality to a second modality can allow for determined positions in the first modality to be tracked or superimposed on or in a reference frame associated with the second modality, thereby providing layers of positional information that can be combined to provide a robust localization system.

In some aspects, the system 300 may be configured to perform one or more localization or localizing techniques. In some implementations, the anatomical space in which a medical instrument can be localized (such as where a pose or shape of the instrument is determined or estimated) may be a 2D or 3D portion of a patient's tracheobronchial airways, vasculature, urinary tract, gastrointestinal tract, or any organ or space accessed via lumens. Various modalities can be implemented to provide images, representations, or models of the anatomical space. For example, an imaging modality can be implemented, which can include, for example, X-ray, fluoroscopy, CT, PET, PET-CT, CT angiography, CBCT, 3DRA, SPECT, MRI, OCT, or ultrasound, among other examples. In some implementations, the imaging modality may be used to capture or acquire images of a patient's anatomy during a preoperative phase of a medical procedure. In some other implementations, the imaging modality may be used to capture or acquire images of a patient's anatomy during an intraoperative phase of the medical procedure.

The systems 302-312 can provide information for generating a graphical interface 314 (also referred to as a “graphical interface (I/F)”) that includes navigation information for navigating an instrument to a target within an anatomy (such as the navigation data 126 (A) or 126 (B) of FIG. 1). For example, the navigation information may include an anatomical map, an estimated position, orientation, and/or shape of the instrument. The navigation information also may include a shape, boundary, eccentricity, texture, and/or position of the target. In some implementations, the graphical user interface 314 or other localization information may be displayed to a user, such as a physician, during a medical procedure to assist the user in performing the procedure. For example, a visualization of a tracked instrument can be superimposed on an anatomical map depicted by the graphical user interface 314 based on position or sensor data associated with the tracked medical instrument.

As shown in FIG. 3, the system 300 can include a support structure 302 (such as a surgical bed or other patient positioning or support platform). For example, the support structure 302 includes a planar surface that contacts and supports the patient. In some implementations, the position of the support structure 302 may be known based on data maintained relating to the position of the support structure 302 within the surgical or procedure environment. In some other implementations, the position of the support structure 302 may be sensed or otherwise determined using one or more markers or an appropriate imaging or positioning modality.

The system 300 can further include a robotic system 304 (such as a robotic cart or other device or system including one or more robotic end effectors). In some implementations, the robotic system 304 may be one example of the robotic system 102 of FIGS. 1 and 2. Data relating to the position or state of robotic arms, actuators, or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration, in some implementations, based on robotic system or cart-set-up procedure (which can have any suitable or desirable scheme).

The system 300 can further include an electromagnetic (EM) sensor system 306, which can include an EM field generator (such as the EM field generator 120 of FIG. 1) and one or more EM sensors. An EM sensor can be associated with a portion of an instrument that is tracked or controlled, such as a distal end (or tip) of the instrument or along a length of the instrument or other elongate member (such as a working channel) disposed in a lumen of the instrument. In some implementations, the EM field generator can be mechanically coupled to the support structure 302 or the robotic system 304 such that registration or association 318 between such systems can be known or determined. In some implementations, the registration 318 between the EM sensor system 306 and the robotic system 304 can be determined through forward kinematics or field generator mount transform information. For example, the field generator can be mounted to the support structure 302 such that the position of the field generator can be known relative to the robotic system positioning frame based on a known relationship between the position of the support structure 302 and the robotic system 304. The EM sensor system 306 can provide instrument pose or path information based on sensor readings associated with the instrument.

The system 300 can further include an optical camera system 308 including one or more cameras or other imaging devices configured to generate images of patient anatomy within a visual field thereof (such as real-time image data) during a surgical procedure. In some implementations, registration 320 between the optical camera system 308 and the EM sensor system 306 can be achieved through identification of features having EM sensor data associated therewith, such as a medical instrument tip, in images generated by the optical camera system 308. The registration 320 can further be based at least in part on hand-eye interaction of the physician when viewing real-time camera images while the EM-sensor-equipped endoscope is navigating in the patient anatomy.

The system 300 can further include a computed tomography (CT) imaging system 310 configured to generate CT images of the patient anatomy, which can be performed preoperatively or intraoperatively. The CT imaging system 310 is generally used for scanning a relatively large volume. In some implementations, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the graphical user interface 314 or for path navigation planning.

In some aspects, the CT imaging system 310 may be registered 326 to the EM sensor system 306 through various techniques. In some implementations, a mechanical structure of the CT imaging system 310 can have a known physical transform or relationship with respect to a mounting position of the EM field generator of the EM sensor system 306. Such known relationship can be used to register the CT image space to the EM sensor space. The connection 328 represents a mapping or relationship between the CT imaging system 310 and an anatomical map depicted by a graphical user interface 314.

The system 300 can further include a fluoroscopy imaging system 312 configured to generate tomographic images (such as real-time X-ray images) of the surgical site. The fluoroscopy imaging system 312 is generally used for scanning a smaller volume compared to the CT imaging system 310. In some implementations, the fluoroscopy imaging system 312 may be one example of the imaging system 122 of FIG. 1. For example, the fluoroscopy imaging system 312 may include a CBCT scanner coupled to a C-arm. In some implementations, the fluoroscopy imaging system 312 may be used with a contrast agent introduced into the anatomy to generate image data representing patient anatomy or instrumentation. In some implementations, the fluoroscopy imaging system 312 may be registered 324 to the CT imaging system 310 using any image processing technique suitable for such registration.

In some aspects, the fluoroscopy imaging system 312 may be registered 332 to the EM sensor system 306 through various techniques. In some implementations, a mechanical structure of the fluoroscopy imaging system 312 (such as the C-arm instrumentation) can have a known physical transform or relationship with respect to a mounting position of the EM field generator of the EM sensor system 306. Such known relationship can be used to register the fluoroscopy image space to the EM sensor space. The connection 330 represents a mapping or relationship between the fluoroscopy imaging system 312 and an anatomical map depicted by the graphical user interface 314.

In the example of FIG. 3, the CT imaging system 310 and fluoroscopy imaging system 312 are illustrated as separated systems. However, in some other implementations, a single imaging system may perform the functions of both the CT imaging system 310 and fluoroscopy imaging system 312.

The position, shape, or orientation of an instrument, such as an endoscope, can be determined using any one or more of the systems 302-312, which can facilitate generation of graphical interface data representing the estimated position or shape of the instrument relative to an anatomical map depicted by the graphical user interface 314. The graphical user interface 314 can be displayed on a display device, such as via the control system 118 or robotic system 102, or another device. In some implementations, the graphical user interface 314 also may indicate a position of a target within the anatomy that has been designated for treatment.

Although the systems 302-312 have been described in a particular order, the operations or functions associated therewith can be performed in different orders. In some implementations, the systems 302-312 can be used in different ways. In some other implementations, registration can occur between different systems and modalities.

In some aspects, one or more of the systems 302-312 may be used to generate the graphical user interface 314 preoperatively or determine a location of one or more targets within an anatomical map depicted by the graphical user interface 314 during a preoperative phase of a medical procedure. As used herein, the terms “skeleton image data” and “skeleton image space” refer to images of an anatomy captured or acquired by an imaging system (such as the CT imaging system 310) during the preoperative phase of a medical procedure. However, a graphical user interface 314 generated using a preoperative CT scan may not accurately reflect the spatial relationship between a medical instrument and a target during an intraoperative phase. For example, changes in the patient's anatomy or the medical environment can cause the spatial relationship between the instrument and the target to deviate from what is depicted in the graphical user interface 314. Example factors that may cause such deviations include EM distortion, poor registration (or mapping) between the sensor data and the image data, outdated preoperative scans, and anatomical deformations, among other examples. Thus, in some other aspects, one or more of the systems 302-312 may be used to determine a location of a medical instrument or position of a target relative to an anatomical map depicted by the graphical user interface 314 during an intraoperative phase of the medical procedure.

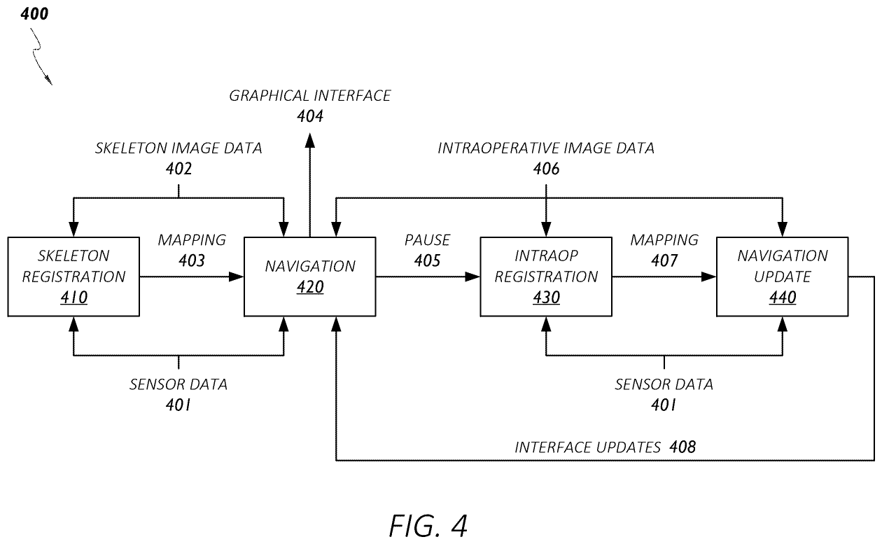

FIG. 4 shows a block diagram of an example navigation system 400, according to some implementations. In some aspects, the navigation system 400 may be implemented by a medical system such as the medical system 100 of FIG. 1. With reference to FIG. 2, the navigation system 400 may be one example of any of the control circuitry 208 or 224. The navigation system 400 is configured to generate or update a graphical interface 404 that depicts or otherwise indicates a spatial relationship between a medical instrument and a target within an anatomy. For example, the graphical interface 404 may be used by the medical system or a user thereof (such as a physician) to navigate the instrument to the target. In some implementations, the graphical interface 404 may be one example of the graphical user interface 314 of FIG. 3.

In some aspects, the navigation system may generate the graphical interface 404 based at least in part on image data 402 (also referred to as “skeleton image data”) acquired via an imaging system (such as the CT imaging system 310 of FIG. 3) and sensor data 401 acquired via a sensor system (such as the EM sensor system 306 of FIG. 3). For example, the skeleton image data 401 may include one or more tomograms captured as a result of a CT scan (such as described with reference to FIGS. 1-3). In some implementations, the skeleton image data 401 may be captured during a preoperative phase of a medical procedure. The sensor data 401 may indicate a pose of the instrument and/or one or more EM sensors disposed in an EM field (such as described with reference to FIGS. 1-3). As used herein, the term “pose” may refer to a position and/or orientation of a sensor or an instrument. In some implementations, the pose of the instrument may be determined based at least in part on sensor data 401 received from a sensor disposed on the instrument (such as at the distal end of an endoscope).

The navigation system 400 includes a skeleton registration component 410, a navigation component 420, an intraoperative registration component 430, and a navigation update component 440. The skeleton registration component 410 is configured to register a coordinate space associated with the sensor data 401 (also referred to as the “sensor space”) to a coordinate space associated with the skeleton image data 402 (also referred to as the “skeleton image space”). As used herein, the term “registration” refers to a mapping between different coordinate spaces. In other words, the skeleton registration component 410 may determine a mapping 403, based at least in part on the sensor data 401 and the skeleton image data 402, that can transform any data point (including a point coordinate or vector) in the sensor space to a respective data point in the image space (such as a transformation matrix).

Various existing techniques can be used to register the skeleton image space with the sensor space, and vice-versa. For example, a number of EM sensors (such as 3 or more EM sensors) may be placed around a patient's body at predetermined positions relative to the anatomy. The skeleton registration component 410 may determine the positions of the EM sensors in an EM field (which represents the sensor space) based on the received sensor data 401. The skeleton registration component 410 also may determine corresponding positions for the EM sensors in the skeleton image space based on their relative locations to known anatomical features depicted by the skeleton image data 402. The skeleton registration component 410 may further determine a mapping 403 (or transformation matrix) that transforms the positions of the EM sensors in the sensor space to their respective positions in the skeleton image space.

The navigation component 420 is configured to generate the graphical interface 404 based on the sensor data 401, the skeleton image data 402, and the mapping 403 between the sensor space and the skeleton image space. The graphical interface 404 may indicate a spatial relationship between the instrument and the target within the anatomy. For example, the graphical interface 404 may include a three-dimensional (3D) model of the anatomy for display on one or more displays associated with the medical system (such as the displays 212 of FIG. 2). In some implementations, the navigation component 420 may render the 3D model based at least in part on the skeleton image data 402. Accordingly, the 3D model of the anatomy may provide a visual indicator of the location of the target within the anatomy. In some aspects, the graphical interface 404 may be used by the medical system or a user (such as a physician) to navigate the instrument to the target during the intraoperative phase of the medical procedure.

In some aspects, the navigation component 420 also may display a pose and/or path of the instrument on the 3D model of the anatomy. For example, the navigation component 420 may determine the pose of the instrument in relation to the anatomy based on the sensor data 401 and the mapping 403 between the sensor space and the skeleton image space. More specifically, the navigation component 420 may use the mapping 403 to transform the pose of the instrument in the sensor space (as indicated by the sensor data 401) to a corresponding pose in the skeleton image space. In some implementations, the navigation component 420 may implement a “fusion” navigation model that combines multiple sensing modalities (such as EM, robotic telemetry, and vision) to determine the instrument pose and/or compensate for any distortions in the sensor data 401. The navigation component 420 may continuously track any changes in the pose or movement of the instrument based on the sensor data 401 and update the graphical interface 404 to reflect the real-time pose of the instrument in relation to the anatomy.

In some aspects, the navigation component 420 may analyze a spatial relationship between the instrument and the target to provide guidance for navigating the instrument within the anatomy. For example, the navigation component 420 may plan a navigation path through the patient's anatomy from the instrument to the target. The navigation path may be rendered or displayed on the graphical interface 404 for the instrument to follow. In some implementations, the navigation component 420 may determine the position of the target in the skeleton image space based at least in part on the skeleton image data. In some other implementations, the navigation component 420 may determine the position of the target in the skeleton image space based at least in part on input provided by a user of the navigation system 400. For example, the user may specify or otherwise input the location of the target on the 3D map of the anatomy.

As described above, images acquired during the preoperative phase may not accurately reflect the spatial relationship between the instrument and the target during the intraoperative phase. For example, changes in the patient's anatomy or the medical environment can cause the spatial relationship between the instrument and target to deviate from what is depicted by the graphical interface 404 at any given time. Example factors that may cause such deviations include EM distortion, poor registration (or mapping 403) between the sensor space and the skeleton image space, outdated skeleton image data 402, and anatomical deformations, among other examples. In some aspects, the medical system may capture updated images of the patient's anatomy via an intraoperative imaging system (such as the fluoroscopy imaging system 312 of FIG. 3) and use the updated image data 406 (also referred to as “intraoperative image data”) to improve or update the depiction of the spatial relationship between the instrument and the target in the graphical interface 404.

In some aspects, the navigation component 420 may be configured to operate in a “navigation mode” or a “re-navigation mode” depending on whether a correction is applied to the graphical interface 404 based on the intraoperative image data 406. For example, the navigation component 420 may operate in the navigation mode while the instrument is initially navigated to a threshold proximity of the target prior to acquiring the intraoperative image data 406. While operating in the navigation mode, the navigation component 420 may use the fusion navigation model, which combines multiple sensing modalities (such as EM, robotic telemetry, and vision), to compensate for any distortions in the sensor data 401. When navigating the instrument past the threshold, the navigation component 420 may be configured to operate in the re-navigation mode. In some aspects, the operating mode of the navigation component 420 may be manually toggled by a user of the navigation system 400. For example, the user may dynamically switch between the navigation mode and the re-navigation mode at any given time during a medical procedure (provided certain preconditions are met). In some implementations, the navigation component 420 may pause 405 navigation or robot motion so that the intraoperative imaging system can be brought to a location proximate the anatomy to capture or acquire the intraoperative image data 406.

The intraoperative registration component 430 is configured to register a coordinate space associated with the intraoperative image data 406 (also referred to as the “intraoperative image space”) with the sensor space responsive to the navigation component 420 signaling a pause 405 in navigation. More specifically, the intraoperative registration component 430 may determine a mapping 407, based at least in part on the sensor data 401 and the intraoperative image data 406, that can transform any data point (including a point coordinate or vector) in the intraoperative image space to a respective data point in the sensor space. In some aspects, the intraoperative registration component 430 may determine the mapping 407 based at least in part on a pose of the instrument in the sensor space and a corresponding instrument pose in the intraoperative image space. In some other aspects, the intraoperative registration component 430 may determine the mapping 407 based on known locations of landmarks (such as fiducial markers or anatomical markers) in both the sensor space and the intraoperative image space.

In some implementations, the intraoperative registration component 430 may determine the pose of the instrument in the intraoperative image space through analysis of the intraoperative image data 406 using one or more image processing techniques. Example suitable image processing techniques include segmentation, machine learning, and statistical analysis, among other examples. As used herein, the term “segmentation” refers to various techniques for partitioning a digital image into groups of voxels (or “image segments”) based on related characteristics or identifying features. In some implementations, the intraoperative registration component 430 may segment the intraoperative image data 406 so that the pose of the instrument can be detected or estimated from the corresponding images (or tomograms).

The intraoperative registration component 430 may determine the pose of the instrument in the sensor space based at least in part on the sensor data 401. Aspects of the present disclosure recognize that some imaging technologies (such as CT or X-rays) may interfere with some sensor technologies (such as EM). Thus, in some implementations, the intraoperative registration component 430 may determine the pose of the instrument in the sensor space based on sensor data 401 captured or acquired while the intraoperative imaging system cannot interfere with the underlying sensor technology (such as while the fluoroscopy imaging system 312 is located outside the EM field of the EM sensor system 306).

The patient is generally breathing while the sensor data 401 is captured but may be forced into a breath-hold while capturing the intraoperative image data 406 (such as to avoid visual artifacts or other inaccuracies in the resulting images). The patient's breathing may cause the instrument pose in the sensor space to differ from the pose in the image space at any given time. In some implementations, the intraoperative registration component 430 may accumulate sensor data 401 over a threshold duration so that time-varying changes in the instrument pose can be detected and compensated in the captured data. For example, the threshold duration may be a predetermined duration configured to span one or more respiratory cycles (such as 12 seconds). As a result, the mapping 407 generated by the intraoperative registration component 430 may compensate for any variations or deviations in the instrument pose due to sensor interference or changes in anatomy.

By compensating for deviations in instrument pose caused by sensor interference when registering the sensor space with the intraoperative image space, the intraoperative registration component 430 may rely on a single sensor modality (such as EM) having data available before any interference is introduced to compensate for any distortions in the sensor data 401. In other words, the intraoperative registration component 430 may not use any of the additional modalities (such as robotic telemetry or vision), that are otherwise used when operating in the navigation mode, to compensate for deviations in instrument pose when determining the mapping 407. In some implementations, the intraoperative registration component 430 may continue to collect data from one or more additional sensing modalities (such as robotics or vision) while determining the mapping 407 and the interface updates 408. More specifically, the intraoperative registration component 430 may use the additional sensor data to detect any distortions in the sensor data 401, for example, to ensure that the sensor data 401 used for registration is undistorted.

The navigation update component 440 is configured to determine one or more interface updates 408 to be applied to the graphical interface 404 based on the intraoperative image data 406 and the mapping 407. In some implementations, the interface updates 408 may include a position of the target transformed from the intraoperative image space to the skeleton image space. In some other implementations, the interface updates 408 may include a pose of the instrument transformed from the intraoperative image space to the sensor space. For example, the navigation update component 440 may determine the target position or the instrument pose in the intraoperative image space through analysis of the intraoperative image data 406 using one or more image processing techniques (such as described above). The navigation update component 440 may further use the mapping 407 to transform the target position or instrument pose in the intraoperative image space to a corrected target position or instrument pose, respectively, in the sensor space. As used herein, the terms “corrected target position” and “corrected instrument pose” refer to the target position and instrument pose that are transformed from the intraoperative image space to the sensor space. The corrected target position and/or the corrected instrument pose in the sensor space may be provided, as the interface updates 408, to the navigation component 420.

Because deviations in instrument pose are compensated when registering the sensor space with the intraoperative image space, the navigation update component 440 also may rely on a single sensor modality (such as EM) to compensate for any distortions in the sensor data 401. In other words, the navigation update component 440 may not use any of the additional modalities (such as robotic telemetry or vision), that are otherwise used when operating in the navigation mode, to compensate for deviations in instrument pose when determining the interface updates 408. As described in greater detail below, any subsequent distortions in sensor data 401 introduced by the imaging system may be compensated by the navigation component 420 when applying the interface updates 408. As such, neither the intraoperative registration component 430 nor the navigation update component 440 may notify a user of any detected distortion when determining the mapping 407 or the interface updates 408.

In some implementations, the navigation component 420 may update the graphical interface 404 based at least in part on the interface updates 408. More specifically, the navigation component 420 may update the spatial relationship between the instrument and the target depicted by the graphical interface 404. In some aspects, the navigation component 420 may update the spatial relationship in the sensor space. For example, the navigation component 420 may use the mapping 403 to transform the position of the target from the skeleton image space to the sensor space. Assuming the physical position of the target remains unchanged from the preoperative phase of the medical procedure to the intraoperative phase, the navigation component 420 may determine a transformation (T^{corr}) that maps the position of the target in the sensor space to the corrected target position indicated by the interface updates 408. The navigation component 420 may apply the same transformation T^{corr} to the sensor data 401 to correct or adjust the spatial relationship between the instrument and the target while operating in the re-navigation mode.

In some other aspects, the navigation component 420 may update the spatial relationship between the instrument and the target in the skeleton space. In some implementations, the navigation component 420 may determine a correction to be applied to the position of the target in the skeleton image space based on the interface updates 408. In such implementations, the navigation component 420 may use the mapping 403 to transform the corrected target position in the sensor space to a corresponding position in the skeleton image space and determine a transformation (tₙ_{^{SKEL}}^{corr}) between the current position of the target in the skeleton image space and the corrected target position. The navigation component 420 may apply the transformation tₙ_{^{SKEL}}^{corr} to the current position of the target (such as by modifying the skeleton image data 402 or the 3D model of the anatomy) to correct or adjust the position of the target relative to the current instrument pose in the graphical interface 404.

In some other implementations, the navigation component 420 may determine a correction to be applied to the instrument pose in the skeleton image space based at least in part on the interface updates 408. In such implementations, the navigation component 420 may apply a correction transformation (T_{S}_{^{SKEL}}^{corr}), based on the transformation tₙ_{^{SKEL}}^{corr} associated with the corrected target position, to real-time sensor data 401 (such as by modifying the sensor data 401) to correct or adjust the pose of the instrument relative to the position of the target in the graphical interface 404. As described above, some imaging technologies may interfere with some sensor technologies. As a result, any sensor data 401 acquired after the intraoperative imaging system is brought within proximity of the anatomy may deviate due to interference caused by the imaging system or further changes to the anatomy. In some implementations, while operating in the re-navigation mode, the navigation component 420 may accumulate the sensor data 401 over a threshold duration (such as 12 seconds) to detect and compensate for any changes or variations in instrument pose over one or more respiratory cycles

Still further, in some implementations, the navigation component 420 may determine corrections to be applied to the instrument pose and the target position in the skeleton image space. In such implementations, the mapping 407 may be determined based on landmarks in the sensor space and the intraoperative image space. For example, the navigation component 420 may determine a correction transformation (T_{S}_{^{Sensor}}^{corr}) that transforms the current instrument pose in the sensor space to the corrected instrument pose and may apply the transformation T_{S}_{^{Sensor}}^{corr} to real-time sensor data 401 (such as by modifying the sensor data 401) to correct or adjust the pose of instrument in the graphical interface 404. Furthermore, the corrected instrument pose in the sensor space may be mapped to the skeleton image space using the mapping 403. The navigation component 420 also may apply the transformation tₙ_{^{SKEL}}^{corr} to the current position of the target (such as by modifying the skeleton image data 402 or the 3D model of the anatomy) to correct or adjust the position of the target in the graphical interface 404.

By compensating for deviations in instrument pose when applying the interface updates 408 and corrections to real-time sensor data 401, the navigation component 420 may rely on a single sensor modality (such as EM) while operating in the re-navigation mode. In other words, the navigation component 420 may not use any of the additional modalities (such as robotic telemetry or vision), that are otherwise used when operating in the navigation mode, to compensate for deviations in instrument pose when operating in the re-navigation mode. In some implementations, the navigation component 420 may continue to collect data from one or more additional sensing modalities while applying the interface updates 408 to the sensor data 401. More specifically, the navigation component 420 may use the additional sensor data to detect any distortions in the sensor data 401, for example, to notify the user of any detected distortions in the re-navigation mode (so that the user can remove any sources of distortion to improve the accuracy of the graphical interface 404).

The updated graphical interface 404 provided by the re-navigation mode may offer many advantages over the existing graphical interface 404 provided by the navigation mode. For example, by updating the spatial relationship between the instrument and the target based on the intraoperative image data 406 acquired during the intraoperative phase, the navigation system 400 can provide more accurate navigation information to the medical system or user. In some aspects, the navigation system 400 may dynamically update the graphical interface 404 based on new intraoperative image data 406. For example, the intraoperative imaging system may be used to scan the anatomy multiple times throughout the intraoperative phase. Each time the anatomy is scanned, the intraoperative registration component 430 may determine a new mapping 407 based on the most recent intraoperative image data 406 and the sensor data 401, and the navigation update component 440 may use the updated mapping 407 to generate new interface updates 408.

The navigation component 420 also may provide additional features in the graphical interface 404 when operating in the re-navigation mode. In some aspects, the navigation component 420 may update the planned navigation path based on the updated patient anatomy depicted by the intraoperative image data 406 and/or the updated spatial relationship between the instrument and the target. For example, the updates may be depicted in contrast (such as with different colors) to the original navigation path. In some implementations, the navigation component 420 may revise the original navigation path (such as by creating new segments or modifying existing segments). In some other implementations, the navigation component 420 may create a new navigation path (also referred to as a “re-navigation path”) from the current instrument pose to the position of the target.

In some other aspects, the navigation component 420 may use the intraoperative image data 406 to supplement or replace at least a portion of the skeleton image data 402. For example, aspects of the present disclosure recognize that by correcting the instrument pose in the skeleton image space based on an instrument pose determined relative to the intraoperative image space, the corrected position of the instrument in the skeleton image space may lie outside of an airway on the 3D model of the anatomy generated using the skeleton image data 402. In some implementations, the navigation component 420 may recreate at least a portion of the 3D model using the intraoperative image data 406. For example, the new 3D model may supplant the original 3D model in the graphical interface 404 (as a whole or in overlapping regions) to provide contemporaneous imaging and anatomical information.

In some other implementations, the graphical interface 404 may display a 3D model of the anatomy based on the skeleton image data 402 and concurrently display another 3D model of the anatomy based on the intraoperative image data 406. For examples, the 3D models may be displayed in contrasting colors or with varying degrees of translucency (which may be user-defined). This may allow the user to assess the degree of deviation or divergences between the 3D models and make commensurate adjustments to workflow or instrument navigation in accordance with their professional judgment. In some implementations, the divergence between the models may be quantified by a set of metrics (such as local deformation field magnitude, percentage overlap, or path angle difference, among other examples) that can be displayed or presented on the graphical interface 404 in various formats (such as heat maps or annotations, among other examples).

As described above, the navigation component 420 may dynamically switch between the navigation mode and the re-navigation mode at any given time. For example, if the user is unsatisfied with the spatial relationship depicted by the graphical interface 404 in the re-navigation mode, or if the user prefers the additional sensing modalities provided by the navigation mode, the user may toggle the navigation component 420 to revert back to the navigation mode at any time. The ability to update the graphical interface 404 with new 3D models of the anatomy may further enhance the functionality for toggling between the navigation mode and the re-navigation mode. For example, displaying multiple 3D models concurrently allows the user to determine which model better reflects the current instrument pose indicated by another sensing modality (such as a camera disposed on the distal end of the instrument). Accordingly, the updated 3D models also may provide a framework for using multiple sensing modalities in the re-navigation mode.

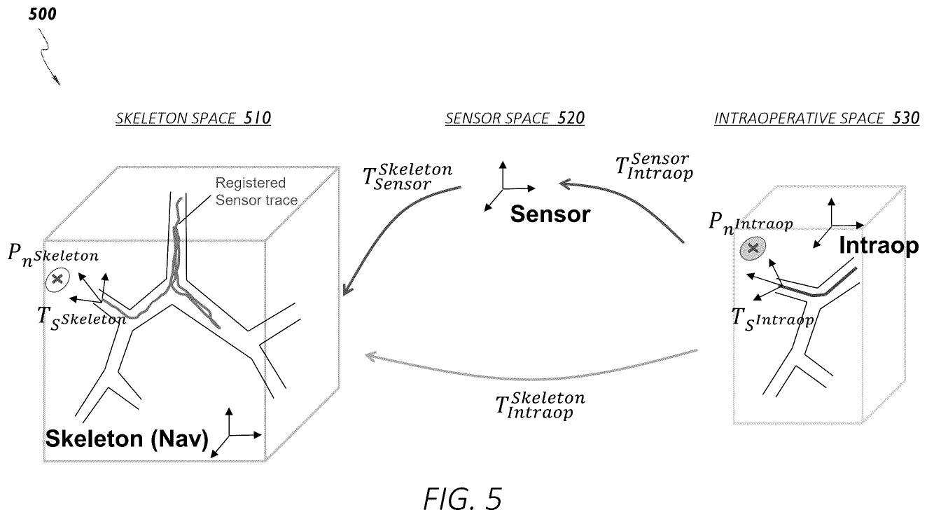

FIG. 5 shows an example spatial relationship 500 between a skeleton image space 510, a sensor space 520, and an intraoperative image space 530. In some implementations, the skeleton image space 510 and the intraoperative image space 530 may be examples of the coordinate spaces associated with the skeleton image data 402 and the intraoperative image data 412, respectively, of FIG. 4. More specifically, FIG. 5 shows a position of a target (Pₙ_{^{Intraop}}) and a pose of an instrument (T_{S}_{^{Intraop}}) in the intraoperative image space 530.

In some aspects, a navigation system (such as the navigation system 400 of FIG. 4) may transform the target position Pₙ_{^{Intraop}} and instrument pose T_{S}_{^{Intraop}} in the intraoperative image space 530 to a target position (Pₙ_{^{Skeleton}}) and an instrument pose (T_{S}_{^{Skeleton}}), respectively, in the skeleton image space 510. As described with reference to FIG. 4, the intraoperative registration component 430 may determine a mapping (T_{Intraop}^{Sensor}) that transforms any data point in the intraoperative image space 530 to a respective data point in the sensor space 520, and the skeleton registration component 410 may determine a mapping (T_{Sensor}^{Skeleton}) that transforms any data point in the sensor space 520 to a respective data point in the skeleton image space 510. In some implementations, the sensor space 520 may be one example of the coordinate space associated with the sensor data 401 of FIG. 4. In some implementations, the mappings T_{Intraop}^{Sensor} and T_{Sensor}^{Skeleton} may be examples of the mappings 407 and 403, respectively, of FIG. 4,

With reference for example to FIG. 4, the navigation update component 440 may apply the mapping T_{Intraop}^{Sensor} to the target position Pₙ_{^{Intraop}} and the instrument pose T_{S}_{^{Intraop}} in the intraoperative image space 530 to transfer the target position and instrument pose to the sensor space 520, and the navigation component 420 may apply the mapping T_{Sensor}^{Skeleton} to further transfer the resulting target position and instrument pose from the sensor space 520 to the skeleton image space 510. Accordingly, the target position Pₙ_{^{Skeleton}} and the instrument pose T_{S}_{^{Skeleton}} in the skeleton image space 510 can be expressed as:

$${P}_{n}^{Skeleton} = {T}_{Sensor}^{Skeleton} * {T}_{Infraop}^{Sensor} * {P}_{n}^{Intraop} \\ {T}_{S}^{Skeleton} = {T}_{Sensor}^{Skeleton} * {T}_{Infraop}^{Sensor} * {T}_{S}^{Intraop}$$

As shown in FIG. 5, a mapping (T_{Intraop}^{Skeleton}) exists between the intraoperative image space 530 and the skeleton image space 510 that can transform any datapoint in the intraoperative image space 530 directly to a data point in the skeleton image space 510, where:

$${T}_{Infraop}^{Skeleton} = {T}_{Sensor}^{Skeleton} * {T}_{Infraop}^{Sensor}$$

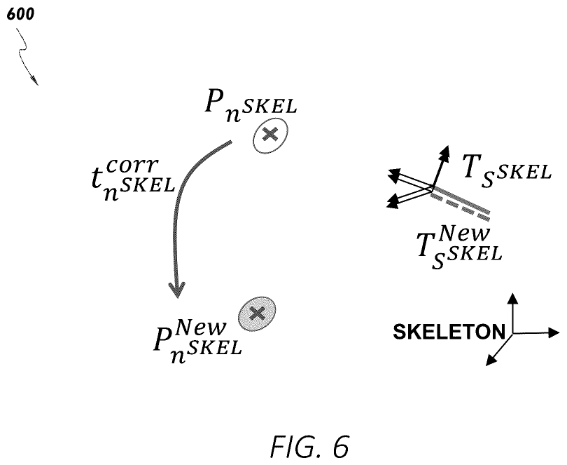

FIG. 6 shows an example update 600 to target position relative to instrument pose. In some implementations, the update 600 may be performed in a skeleton image space (such as the coordinate space associated with the skeleton image data 402 of FIG. 4). More specifically, FIG. 6 shows a current position of a target (Pₙ_{^{SKEL}}) and a current pose of an instrument (T_{S}_{^{SKEL}}), as well as a corrected position of the target (Pₙ_{^{SKEL}}^{New}) and a corrected pose of the instrument (T_{S}_{^{SKEL}}^{New}), in the skeleton image space.

In some aspects, a navigation system (such as the navigation system 400 of FIG. 4) may determine a correction (tₙ_{^{SKEL}}^{corr}) to be applied to the current target position Pₙ_{^{SKEL}} based on the corrected target position pₙ_{^{SKEL}}^{New}. In the example of FIG. 6, the navigation system may assume that the current instrument pose T_{S}_{^{SKEL}} represents or otherwise coincides with the corrected instrument pose T_{S}_{^{SKEL}}^{New}. Thus, the correction tₙ_{^{SKEL}}^{corr} represents a transformation between the current target position Pₙ_{^{SKEL}} and the corrected target position Pₙ_{^{SKEL}}^{New}, where:

$${t}_{{n}^{SKEL}}^{corr} = {P}_{{n}^{SKEL}}^{New} - {P}_{n}^{SKEL} \qquad ( 1 )$$

As described with reference to FIG. 4, the navigation component 420 may apply the correction tₙ_{^{SKEL}}^{corr} to the skeleton image data 402 or the 3D model of the anatomy to update the spatial relationship between the instrument and the target depicted by the graphical interface 404. Among other advantages, the correction tₙ_{^{SKEL}}^{corr} can be applied once and paired with subsequent sensor data (even in the absence of sensor data, such as due to EM dropout) without requiring continuous updates.

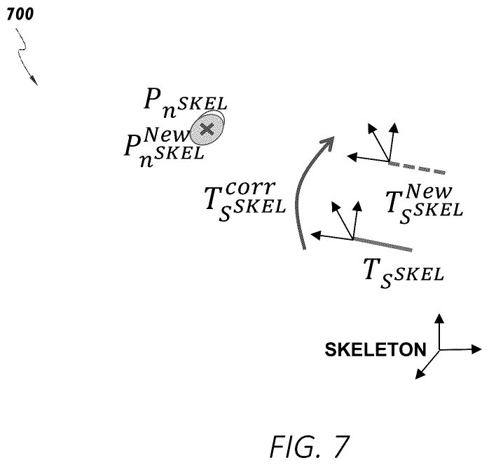

FIG. 7 shows another example update 700 to instrument pose relative to target position. In some implementations, the update 700 may be performed in a skeleton image space (such as the coordinate space associated with the skeleton image data 402 of FIG. 4). More specifically, FIG. 7 shows a current position of a target (Pₙ_{^{SKEL}}) and a current pose of an instrument (T_{S}_{^{SKEL}}), as well as a corrected position of the target (Pₙ_{^{SKEL}}^{New}) and a corrected pose of the instrument (T_{S}_{^{SKEL}}^{New}), in the skeleton image space.

In some aspects, a navigation system (such as the navigation system 400 of FIG. 4) may determine a correction (T_{S}_{^{SKEL}}^{corr}) to be applied to the current instrument pose T_{S}_{^{SKEL}} based on the transformation tₙ_{^{SKEL}}^{corr}, between the current target position Pₙ_{^{SKEL}} and the corrected target position Pₙ_{^{SKEL}}^{New}, shown in Equation 1 (as described with reference to FIG. 6). In the example of FIG. 7, the navigation system may assume that the current target position Pₙ_{^{SKEL}} represents or otherwise coincides with the corrected target position Pₙ_{^{SKEL}}^{New}, the correction T_{S}_{^{SKEL}}^{corr} represents a transformation matrix between the current instrument pose T_{S}_{^{SKEL}} and the corrected instrument pose T_{S}_{^{SKEL}}^{New}, where:

$${T}_{{S}^{SKEL}}^{corr} = [ \begin{matrix}I & - {t}_{{n}^{SKEL}}^{corr} \\ 0 & 0\end{matrix} ] \qquad ( 2 )$$

where I is the identity matrix.

As described with reference to FIG. 4, the navigation component 420 may apply the correction T^{corr} to the sensor data 401 or the 3D model of the anatomy to update the spatial relationship between the instrument and the target depicted by the graphical interface 404. Among other advantages, applying the correction T^{corr} to the sensor data 401 allows the navigation system to compensate for deviations in instrument pose that may be caused by sensor interference or changes in anatomy (such as described with reference to FIG. 4).

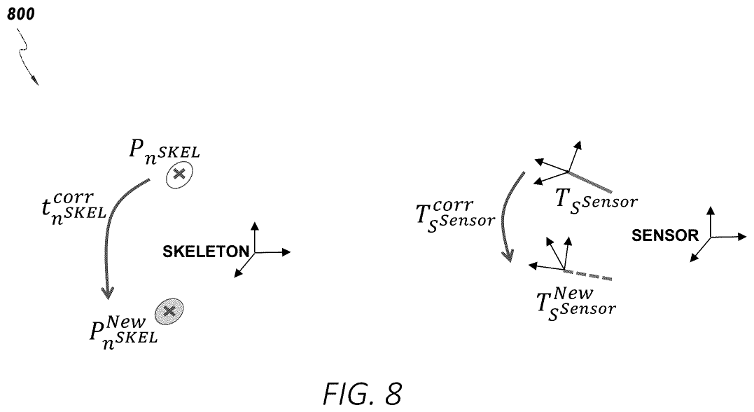

FIG. 8 shows example corrections 800 to instrument pose and target position in their respective coordinate spaces and updates to a spatial relationship between the instrument and the target in a common coordinate space (such as a skeleton image space). In the example of FIG. 8, the update to the instrument pose is depicted in the sensor space but can be mapped to the skeleton image space using any of the techniques described with reference to FIGS. 4 and 5 (such as via the mapping 403). In some implementations, the skeleton image space and the sensor space may be examples of the coordinate spaces associated with the skeleton image data 402 and sensor data 401, respectively, of FIG. 4. More specifically, FIG. 8 shows a current position of a target (Pₙ_{^{SKEL}}) and a corrected position of the target (Pₙ_{^{SKEL}}^{New}) in the skeleton image space, as well as a current pose of an instrument (T_{S}_{^{Sensor}}) and a corrected pose of the instrument (T_{S}_{^{Sensor}}^{New}) in the sensor space.

In some aspects, a navigation system (such as the navigation system 400 of FIG. 4) may determine a correction (T_{S}_{^{Sensor}}^{corr}) to be applied to the current instrument pose T_{S}_{^{Sensor}} and a correction (tₙ_{^{SKEL}}^{corr}) to be applied to the current target position Pₙ_{^{SKEL}}. As described with reference to FIG. 4, the corrections T_{S}_{^{Sensor}}^{corr} and tₙ_{^{SKEL}}^{corr} rely on a mapping 407 between the intraoperative image space and the sensor space that is determined based on landmarks in both coordinate spaces (rather the instrument pose alone). The correction tₙ_{^{SKEL}}^{New} can be determined based on the current target position Pₙ_{^{SKEL}} and the corrected target position Pₙ_{^{SKEL}}^{New}, according to Equation 1 (as described with reference to FIG. 6). Similarly, the correction T_{S}_{^{Sensor}}^{corr} can be determined as a transformation between the current instrument pose T_{S}_{^{Sensor}} and the corrected instrument pose T_{S}_{^{Sensor}}^{New}Sensor, where:

$${T}_{{S}^{Sensor}}^{corr} = {[ {T}_{S}^{Sensor} ]}^{- 1} * {T}_{{S}^{Sensor}}^{New} \qquad ( 3 )$$

As described with reference to FIG. 4, the navigation component 420 may apply the corrections T_{S}_{^{Sensor}}^{corr} and tₙ_{^{SKEL}}^{corr} to the sensor data 401 and the skeleton image data 402, respectively, or the 3D model of the anatomy, to update the spatial relationship between the instrument and the target depicted by the graphical interface 404. For example, the navigation component 420 may convert the current instrument pose T_{S}_{^{Sensor}} to a respective instrument pose (T_{S}_{^{SKEL}}^{New}) in the skeleton image space by applying a mapping (T_{Sensor}^{SKEL}) that transforms any data point in the sensor space to a respective data point in the skeleton image space (such as the mapping 403), where:

$${T}_{{S}^{SKEL}}^{New} = {T}_{Sensor}^{SKEL} * {T}_{{S}^{Sensor}}^{corr} * {T}_{S}^{Sensor}$$

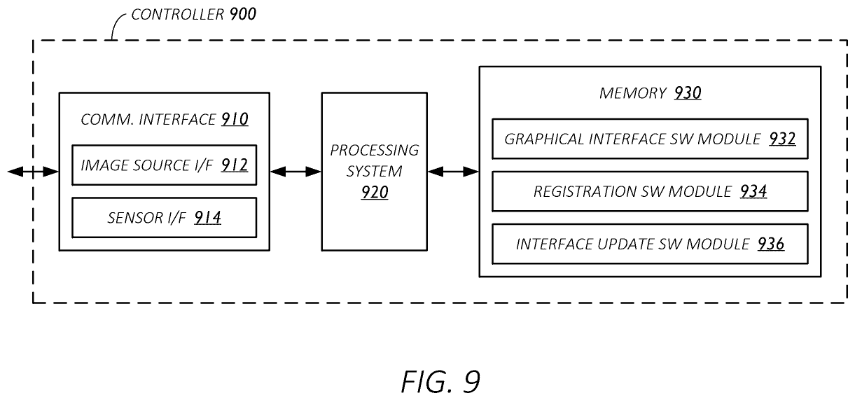

FIG. 9 shows a block diagram of an example controller 900 for a medical system, according to some implementations. In some implementations, the controller 900 may be one example of the navigation system 400 of FIG. 4. More specifically, the controller 900 is configured to generate and/or update a graphical interface that depicts or otherwise indicates a spatial relationship between an instrument and a target within an anatomy.

The controller 900 includes a communication interface 910, a processing system 920, and a memory 930. The communication interface 910 is configured to communicate with one or more components of the medical system. More specifically, the communication interface 910 includes an image source interface (I/F) 912 for communicating with one or more image sources (such as the CT imaging system 310 and/or the fluoroscopy imaging system 312 of FIG. 3) and a sensor interface (I/F) 914 for communicating with one or more sensors (such as the EM sensor system 306 of FIG. 3). In some implementations, the image source I/F 912 may receive image data captured by an imaging system external to the anatomy while the instrument is disposed within the anatomy. In some implementations, the sensor I/F 914 may receive sensor data via one or more sensors associated with a sensor system, where the one or more sensors include at least a first sensor disposed on the instrument.

The memory 930 may include a non-transitory computer-readable medium (including one or more nonvolatile memory elements, such as EPROM, EEPROM, Flash memory, or a hard drive, among other examples) that may store the following software (SW) modules: a graphical interface SW module 932 to generate a graphical interface depicting a spatial relationship between the instrument and the target; a registration SW module 934 to determine a mapping between a first coordinate space associated with the imaging system and a second coordinate space associated with the sensor system based at least in part on the image data and the sensor data; and an interface update SW module 936 to update the graphical interface to depict an updated spatial relationship between the instrument and the target based at least in part on the mapping between the first coordinate space and the second coordinate space. Each of the software modules 932-936 includes instructions that, when executed by the processing system 920, causes the controller 900 to perform the corresponding functions.

The processing system 920 may include any suitable one or more processors capable of executing scripts or instructions of one or more software programs stored in the controller 900 (such as in the memory 930). For example, the processing system 920 may execute the graphical interface SW module 932 to generate a graphical interface depicting a spatial relationship between the instrument and the target. The processing system 920 also may execute the registration SW module 934 to determine a mapping between a first coordinate space associated with the imaging system and a second coordinate space associated with the sensor system based at least in part on the image data and the sensor data. The processing system 920 may further execute the interface update SW module 936 to update the graphical interface to depict an updated spatial relationship between the instrument and the target based at least in part on the mapping between the first coordinate space and the second coordinate space.

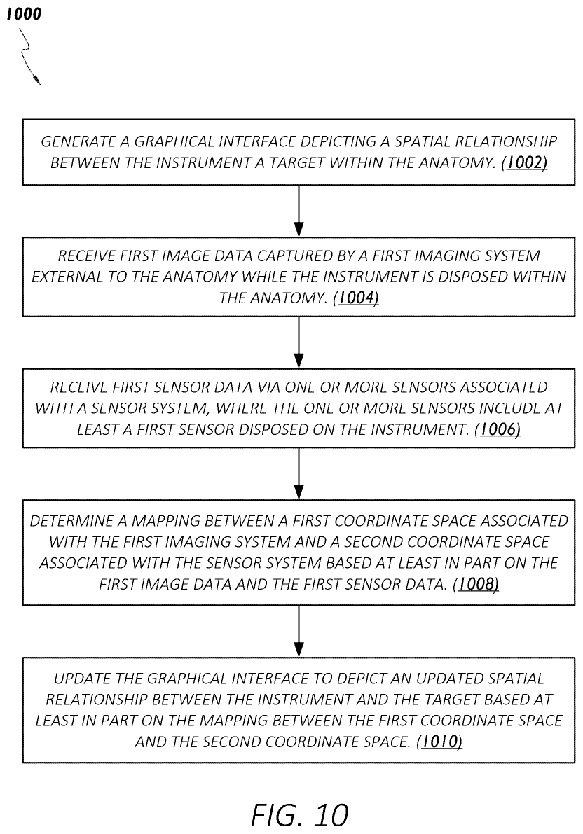

FIG. 10 shows an illustrative flowchart depicting an example operation 1000 for navigating an instrument within an anatomy, according to some implementations. In some implementations, the example operation 1000 may be performed by a controller for a medical system such as the controller 900 of FIG. 9 or the navigation system 400 of FIG. 4.

The controller may generate a graphical interface depicting a spatial relationship between the instrument and a target within the anatomy (1002). The controller may receive first image data captured by a first imaging system external to the anatomy while the instrument is disposed within the anatomy (1004). In some implementations, the first imaging system may be a cone beam computed tomography (CBCT) system. The controller may receive first sensor data via one or more sensors associated with a sensor system, where the one or more sensors include at least a first sensor disposed on the instrument (1006). The controller may determine a mapping between a first coordinate space associated with the first imaging system and a second coordinate space associated with the sensor system based at least in part on the first image data and the first sensor data (1008). The controller may further update the graphical interface to depict an updated spatial relationship between the instrument and the target based at least in part on the mapping between the first coordinate space and the second coordinate space (1010).

In some aspects, the generating of the graphical interface may include receiving second image data via a second imaging system external to the anatomy, receiving second sensor data via the first sensor disposed on the instrument, determining a mapping between the second coordinate space and a third coordinate space associated with the second imaging system based at least in part on the second image data and the second sensor data, and determining the spatial relationship between the instrument and the target based at least in part on the mapping between the second coordinate space and the third coordinate space.

In some implementations, the updating of the graphical interface may include determining a position of the target in the third coordinate space based on the second image data, determining a position of the target in the first coordinate space based on the received first image data, determining a transformation that transforms the position of the target in the first coordinate space to the position of the target in the third coordinate space based on the mapping between the first coordinate space and the second coordinate space and the mapping between the second coordinate space and the third coordinate space, and applying a correction associated with the transformation to subsequent sensor data received via the first sensor disposed on the instrument.

In some implementations, the controller may further determine a position of the target in the first coordinate space based on the first image data, transform the position of the target from the first coordinate space to the third coordinate space based on the mapping between the first coordinate space and the second coordinate space and the mapping between the second coordinate space and the third coordinate space, and determine the updated spatial relationship between the instrument and the target based at least in part on the position of the target in the third coordinate space.

In some aspects, the generating of the graphical interface may further include generating a first model of the anatomy based on the second image data and displaying the spatial relationship between the instrument and the target on the first model of the anatomy. In some implementations, the updating of the graphical interface may include displaying the updated spatial relationship between the instrument and the target on the first model of the anatomy. In some implementations, the updating of the graphical interface may include reconstructing at least a portion of the first model of the anatomy based on the first image data. In some other implementations, the updating of the graphical interface may include generating a second model of the anatomy based on the first image data and displaying the updated spatial relationship between the instrument and the target on the second model of the anatomy.

In some aspects, the generating of the graphical interface may include determining a path for navigating the instrument within the anatomy based at least in part on the second image data. In some implementations, the updating of the graphical interface may include updating the path for navigating the instrument within the anatomy based at least in part on the first image data.

In some aspects, the one or more sensors may further include a plurality of sensors disposed at known locations in each of the first coordinate space and the second coordinate space, where the mapping between the first coordinate space and the second coordinate space is determined based on the first sensor data received via the plurality of sensors.

In some implementations, the updating of the graphical interface may include determining a pose of the instrument in the first coordinate space based on the first sensor data received via the first sensor, determining a corrected pose of the instrument in the second coordinate space based on the first image data, transforming the corrected pose of the instrument from the second coordinate space to the first coordinate space based on the mapping between the first coordinate space and the second coordinate space, determining a correction transformation that transforms the pose of the instrument in the first coordinate space to the corrected pose of the instrument in the first coordinate space, and applying the correction transformation to subsequent sensor data received via the first sensor disposed on the instrument.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method of navigating an instrument within an anatomy, comprising:
generating a graphical interface depicting a spatial relationship between the instrument and a target within the anatomy;
receiving first image data captured by a first imaging system external to the anatomy while the instrument is disposed within the anatomy;
receiving first sensor data via one or more sensors associated with a sensor system, the one or more sensors including at least a first sensor disposed on the instrument;
determining a mapping between a first coordinate space associated with the first imaging system and a second coordinate space associated with the sensor system based at least in part on the first image data and the first sensor data; and
updating the graphical interface to depict an updated spatial relationship between the instrument and the target based at least in part on the mapping between the first coordinate space and the second coordinate space.

2. The method of claim 1, wherein the first imaging system is a cone beam computed tomography (CBCT) system.

3. The method of claim 1, wherein the generating of the graphical interface comprises:
receiving second image data via a second imaging system external to the anatomy;
receiving second sensor data via the first sensor disposed on the instrument;
determining a mapping between the second coordinate space and a third coordinate space associated with the second imaging system based at least in part on the second image data and the second sensor data; and
determining the spatial relationship between the instrument and the target based at least in part on the mapping between the second coordinate space and the third coordinate space.

4. The method of claim 3, wherein the updating of the graphical interface comprises:
determining a position of the target in the third coordinate space based on the second image data;
determining a position of the target in the first coordinate space based on the received first image data;
determining a transformation that transforms the position of the target in the first coordinate space to the position of the target in the third coordinate space based on the mapping between the first coordinate space and the second coordinate space and the mapping between the second coordinate space and the third coordinate space; and
applying a correction associated with the transformation to subsequent sensor data received via the first sensor disposed on the instrument.

5. The method of claim 3, further comprising:
determining a position of the target in the first coordinate space based on the first image data;
transforming the position of the target from the first coordinate space to the third coordinate space based on the mapping between the first coordinate space and the second coordinate space and the mapping between the second coordinate space and the third coordinate space; and
determining the updated spatial relationship between the instrument and the target based at least in part on the position of the target in the third coordinate space.

6. The method of claim 3, wherein the generating of the graphical interface further comprises:
generating a first model of the anatomy based on the second image data; and
displaying the spatial relationship between the instrument and the target on the first model of the anatomy.

7. The method of claim 6, wherein the updating of the graphical interface comprises:
displaying the updated spatial relationship between the instrument and the target on the first model of the anatomy.

8. The method of claim 6, wherein the updating of the graphical interface comprises:
reconstructing at least a portion of the first model of the anatomy based on the first image data.

9. The method of claim 6, wherein the updating of the graphical interface comprises:
generating a second model of the anatomy based on the first image data; and
displaying the updated spatial relationship between the instrument and the target on the second model of the anatomy.

10. The method of claim 3, wherein the generating of the graphical interface comprises:
determining a path for navigating the instrument within the anatomy based at least in part on the second image data.

11. The method of claim 10, wherein the updating of the graphical interface comprises:
updating the path for navigating the instrument within the anatomy based at least in part on the first image data.

12. The method of claim 1, wherein the one or more sensors further include a plurality of sensors disposed at known locations in each of the first coordinate space and the second coordinate space, the mapping between the first coordinate space and the second coordinate space being determined based on the first sensor data received via the plurality of sensors.

13. The method of claim 12, wherein the updating of the graphical interface comprises:
determining a pose of the instrument in the first coordinate space based on the first sensor data received via the first sensor;
determining a corrected pose of the instrument in the second coordinate space based on the first image data;
transforming the corrected pose of the instrument from the second coordinate space to the first coordinate space based on the mapping between the first coordinate space and the second coordinate space;
determining a correction transformation that transforms the pose of the instrument in the first coordinate space to the corrected pose of the instrument in the first coordinate space; and
applying the correction transformation to subsequent sensor data received via the first sensor disposed on the instrument.

14. A controller for a medical system, comprising:
a processing system; and
a memory storing instructions that, when executed by the processing system, cause the controller to:
generate a graphical interface depicting a spatial relationship between the instrument and a target within the anatomy;
receive first image data captured by a first imaging system external to the anatomy while the instrument is disposed within the anatomy;
receive first sensor data via one or more sensors associated with a sensor system, the one or more sensors including at least a first sensor disposed on the instrument;
determine a mapping between a first coordinate space associated with the first imaging system and a second coordinate space associated with the sensor system based at least in part on the first image data and the first sensor data; and
update the graphical interface to depict an updated spatial relationship between the instrument and the target based at least in part on the mapping between the first coordinate space and the second coordinate space.

15. The controller of claim 14, wherein the first imaging system is a cone beam computed tomography (CBCT) system.

16. The controller of claim 14, wherein the generating of the graphical interface comprises:
receiving second image data via a second imaging system external to the anatomy;
receiving second sensor data via the first sensor disposed on the instrument;
determining a mapping between the second coordinate space and a third coordinate space associated with the second imaging system based at least in part on the second image data and the second sensor data; and
determining the spatial relationship between the instrument and the target based at least in part on the mapping between the second coordinate space and the third coordinate space.

17. The controller of claim 16, wherein the updating of the graphical interface comprises:
determining a position of the target in the third coordinate space based on the second image data;
determining a position of the target in the first coordinate space based on the received first image data;
determining a transformation that transforms the position of the target in the first coordinate space to the position of the target in the third coordinate space based on the mapping between the first coordinate space and the second coordinate space and the mapping between the second coordinate space and the third coordinate space; and
applying a correction associated with the transformation to subsequent sensor data received via the first sensor disposed on the instrument.

18. The controller of claim 16, wherein execution of the instructions further causes the controller to:
determine a position of the target in the first coordinate space based on the first image data;
transform the position of the target from the first coordinate space to the third coordinate space based on the mapping between the first coordinate space and the second coordinate space and the mapping between the second coordinate space and the third coordinate space; and
determine the updated spatial relationship between the instrument and the target based at least in part on the position of the target in the third coordinate space.

19. The controller of claim 14, wherein the one or more sensors further include a plurality of sensors disposed at known locations in each of the first coordinate space and the second coordinate space, the mapping between the first coordinate space and the second coordinate space being determined based on the first sensor data received via the plurality of sensors.

20. The controller of claim 19, wherein the updating of the graphical interface comprises:
determining a pose of the instrument in the first coordinate space based on the first sensor data received via the first sensor;
determining a corrected pose of the instrument in the second coordinate space based on the first image data;
transforming the corrected pose of the instrument from the second coordinate space to the first coordinate space based on the mapping between the first coordinate space and the second coordinate space;
determining a correction transformation that transforms the pose of the instrument in the first coordinate space to the corrected pose of the instrument in the first coordinate space; and
applying the correction transformation to subsequent sensor data received via the first sensor disposed on the instrument.
